(19) United States
(12) Patent Application Publication
(10) Pub. No.: US 2008/0154093 A1
(43) Pub. Date: Jun. 26, 2008

(54) Capsule Type Endoscope with an Insertion Tube

(75) Inventors: Nam Kyu Cho, Gyeonggi-do (KR); Dae Sung Lee, Gyeonggi-do (KR); Kyu Sik Shin, Seoul (KR); Don Haeng Lee, Seoul (KR)

Correspondence Address: THOMAS, KAYDEN, HORSTEMEYER & RISLEY, LLP, 600 GALLERIA PARKWAY, S.E., STE 1500, ATLANTA, GA, 30339-5994, US

(73) Assignee: KOREA ELECTRONICS TECHNOLOGY INSTITUTE, Gyeonggi-do (KR)

(21) Appl. No.: 11/941,476

(22) Filed: Nov. 16, 2007

(30) Foreign Application Priority Data
Dec. 26, 2006 (KR) 10-2006-0134134

(51) Int. Cl. A61B 1/01

(52) U.S. Cl. 600/114

(57) ABSTRACT

Disclosed is a capsule type endoscope with an insertion tube, which is capable of improving diagnosis and treatment of a patient's disease by attaching manipulation wires, an electric power line, and a signal line to a conventional capsule type endoscope. A specific embodiment of the present invention comprises and is characterized by a capsule containing a photographic system for acquiring images from the internal cavities of the human body; at least one manipulation wire connected to the capsule; an electric power line and a signal line connected to the capsule; an insertion tube for protecting the manipulation wire, the electric power line and the signal line; and a connection section for connecting the capsule with the insertion tube.

### BACKGROUND OF THE INVENTION

1. Field of the Invention

The present invention relates to a capsule type endoscope with an insertion tube; and, more particularly, one that is capable of improving diagnosis and treatment of a patient's disease by attaching manipulation wires, an electric power line, and a signal line to a conventional capsule type endoscope.

2. Background of the Related Art

The endoscope is one of the instruments used for visualizing the interior of the human body. However, endoscopes with a large diameter cannot display the small intestine in its entirety and may cause pain and rejection symptoms to the patient. Recently developed tubeless capsule type endoscopes are meant to perform the diagnostic or therapeutic functions inside the esophagus, stomach, and intestines, while reducing pain and rejection symptoms involved.

Once the pill-shaped capsule type endoscope has been swallowed with water, it undergoes a random upward and downward movement caused by peristalsis in the esophagus and then travels on through the gastrointestinal tract. In the mean time, the patient's anatomical information is transmitted via various sensors attached to the body and/or a wireless communication means.

FIG. 1 shows the construction of a capsule type endoscope 100, which has been published as Registered Utility Model No. 20-0344193 (Korea).

The capsule type endoscope 100 is primarily a photographic system for acquiring images from the internal cavities of the body such as the Gastrointestinal (GI) tract. The photographic system comprises an illumination section 110 containing at least one light source such as a white LED; a signal processing section 120 consisting of a CMOS camera; and an optical sensor section 130 for focal adjustment of images to be processed by the signal processing section 120.

The illumination section 110 irradiates the internal cavities of the body through an optical window 140. The capsule type endoscope 100 also comprises a signal transceiver portion 150 consisting of a transmitter and antenna for transmitting video signals obtained by the CMOS camera of the signal processing section 120 and a silver oxide battery unit 160 for supplying power to electrical elements of the capsule type endoscope 100. The capsule type endoscope 100 has a number of advantages such as convenience, simplicity, and pain-free application.

However, the capsule type endoscope 100 has disadvantages as well. The capsule type endoscope 100 passes through the esophagus rather too quickly and then free-falls upon reaching the stomach due to the large size of the stomach compared with the tiny pill-sized capsule type endoscope 100. Besides, the pace of its movement from the lower portion of the stomach to the duodenum varies greatly for different people. Such a random or fast movement of the capsule type endoscope 100 limits the possibility of intensive observation of suspected areas. Other disadvantages include its limited operational time due to use of a built-in battery unit 160 and unreliability of precise transmission from the signal transceiver portion 150 to the outside wireless communications receiver.

### SUMMARY OF THE INVENTION

An embodiment of the present invention is directed to a mechanism whereby a manipulation wire connected to the capsule enables the operator to steer and manipulate the endoscope according to his/her needs.

In another embodiment of the invention, the electric power line connected to the capsule extends the operational time of the endoscope as long as required.

In still another embodiment of the invention, a signal line is likewise connected to the capsule to guarantee precise transmission of video signals obtained.

The present invention relates to a capsule type endoscope with an insertion tube, comprising:
a capsule containing a photographic system for acquiring images of the interior of the human body; at least one manipulation wire connected to the above-mentioned capsule;
an electric power line and a signal line connected to the capsule;
an insertion tube for protecting the above-mentioned manipulation wire, electric power line, and signal line; and
a connection section for attaching the capsule to the insertion tube.

### BRIEF DESCRIPTION OF THE DRAWINGS

The appended drawings illustrate currently preferred embodiments of the present invention except for FIG. 1.

FIG. 1 illustrates the construction of a conventional capsule type endoscope.

FIG. 2 shows the construction of the capsule type endoscope with an insertion tube as an embodiment of the present invention.

FIG. 3 illustrates a clinical application of the capsule type endoscope with an insertion tube as an embodiment of the present invention.

FIG. 4 is a view showing the structure of another embodiment of the capsule type endoscope with an insertion tube.

FIG. 5 shows still another embodiment of the capsule type endoscope with an insertion tube.

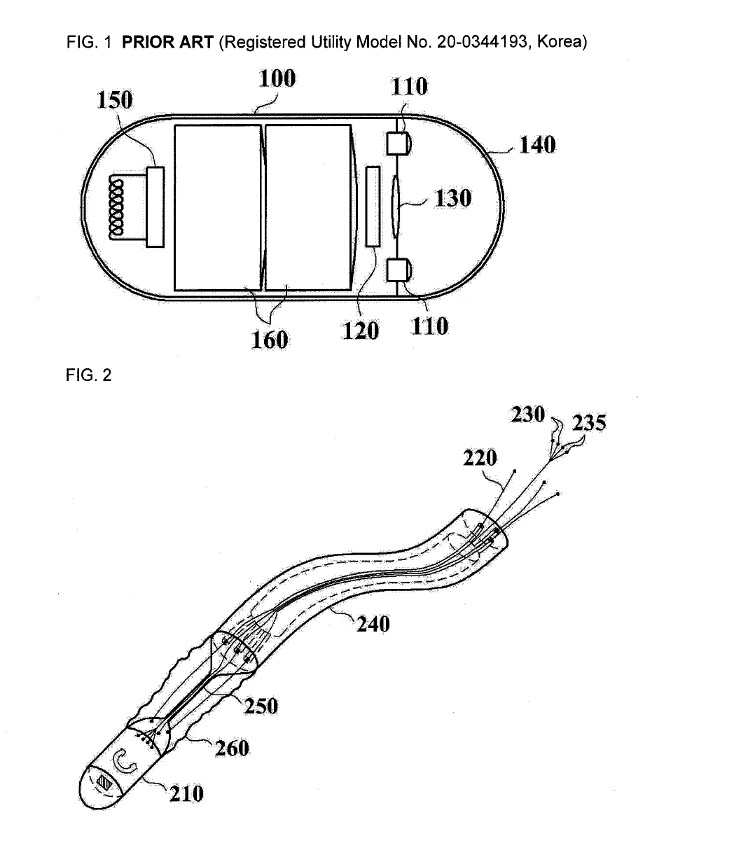

### DETAILED DESCRIPTION OF THE PREFERRED EMBODIMENTS

It should be noted that the terms and words used in the specification and claims pertaining to the present invention are best understood when interpreted in the context of the technological concept and scope of the present invention.

The constructions as illustrated and described in the figures and specification herein are merely representative of the currently preferred embodiments of the present invention. These preferred embodiments, therefore, should not lead one to doubt the existence of diverse equivalent or modified substitutes for them at the time of filing the present application, nor do they represent the entire technological concept and scope of the present invention.

Hereinafter, the preferred embodiments of the present invention will be described in more detail.

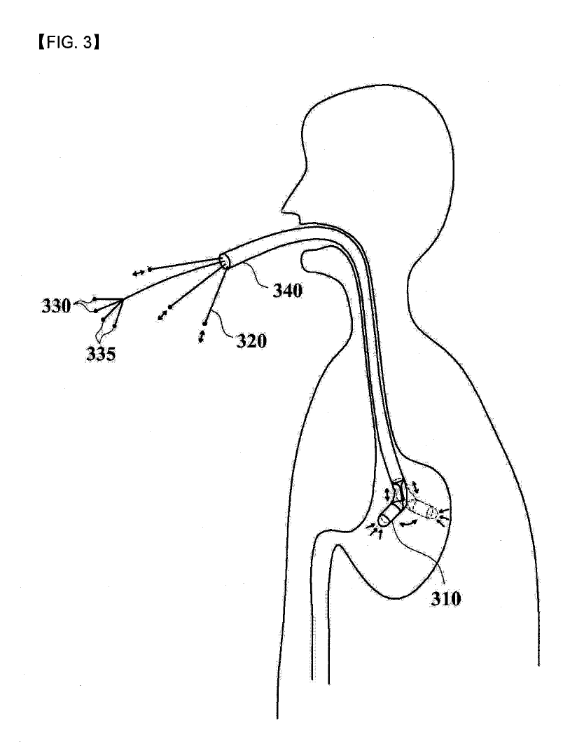

FIG. 2 shows the construction of the capsule type endoscope with an insertion tube as an embodiment of the present invention.

As illustrated in FIG. 2, the above-identified embodiment comprises: a capsule 210, a manipulation wire 220, an electric power line 235, an insertion tube 240, a connection section 250, and a flexible connection tube 260.

The capsule 210, though not shown in detail, comprises an illumination section, a signal processing section, an optical sensor section, an optical window, and a signal transmission section. The functions of the respective components of the capsule 210 are largely the same as those of a conventional capsule type endoscope. Their difference lies in their power supply and signal transmission mechanisms. And the absence of a battery unit and antenna in the capsule 210 may result in a smaller size than that of a conventional capsule type endoscope.

The manipulation wire 220 connected to the rear end of the capsule 210 can control the position and direction of the endoscope, thus making possible close observation of suspected areas from various angles and perspectives for a sustained period of time.

The electric power line 230 supplying electric power to the capsule 210 ensures a sufficient operational time.

The signal line 235 makes an outside wireless communications receiver irrelevant and allows for precise transmission of video signals.

The electric power line 230 and the signal line 235 are combined into a single strand to minimize the space they occupy.

The insertion tube 240 protects the manipulation wire 220, the electric power line 230, and the signal line 235. The insertion tube 240 should be made of flexible material, such as silicon or polyurethane, to ensure adequate maneuverability required to obtain sufficient visual information inside the tortuous intestinal lumen.

The connection section 250, where the capsule 210 is connected with the insertion tube 240, is slim and flexible so that it can be easily bent and oriented according to the control signal sent via the manipulation wire 220.

The flexible connection tube 260 connects the capsule 210 with the insertion tube 240 and protects the connection section 250 and the manipulation wire 220. The flexible connection tube 260 shaped like a corrugated tube is made of flexible material to facilitate rotation and tilting of the capsule.

FIG. 3 is an illustrative view showing a clinical application of the capsule type endoscope with an insertion tube in a preferred embodiment of the present invention.

As illustrated in the figure, the operator can achieve a dual-axis movement by applying an appropriate pulling force to any one of the plurality of manipulation wires 320 attached around the rear end of the capsule 310 at intervals of 90 or 120 degrees.

The operator can control navigation by pushing or pulling the insertion tube 340 while pulling the manipulation wire 320 as required for steering.

Whereas the operational time of a conventional capsule type endoscope was limited by battery life, that of an embodiment of the present invention, which has an electric power line 330, is virtually unlimited as far as power supply is concerned.

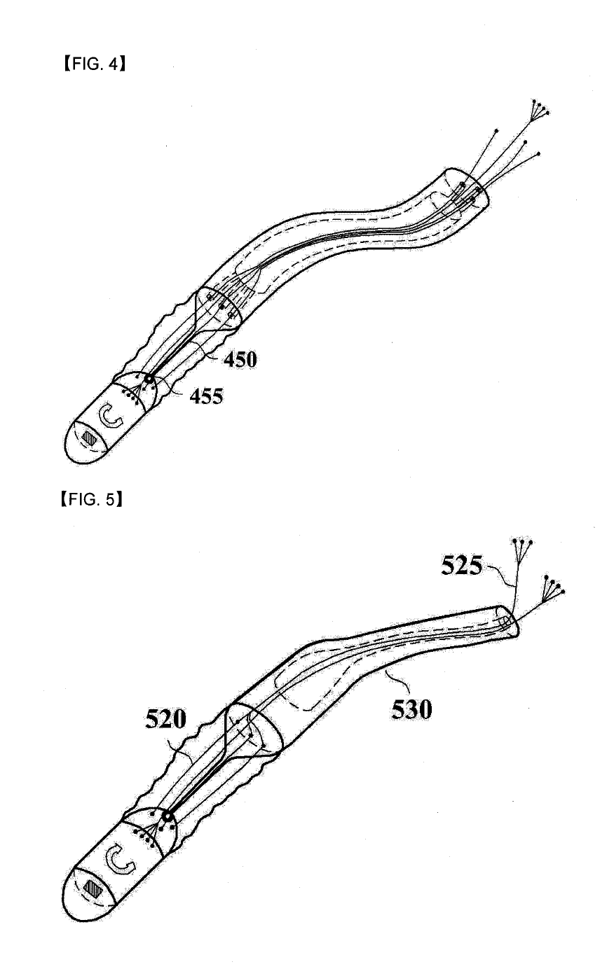

FIG. 4 illustrates the construction of another preferred embodiment of the present invention.

As shown in FIG. 4, this embodiment is characterized by the ball swivel system 455 at the contact point of the connection section 450 and the capsule. This allows for a higher standard of precision and reliability in the control of the rotation and tilting angle required to obtain images from a variety of angles and perspectives in its diagnostic or therapeutic application.

FIG. 5 shows still another preferred embodiment of the present invention.

In addition to the ball swivel system, this embodiment features muscle wire, which is a kind of shape memory alloy whose length can be changed by an electrical signal. The muscle wires 520 replace the manipulation wires for the length from the rear end of the capsule to the insertion tube 530. The rest of the manipulation wires are replaced by a single strand of leading wire 525. This embodiment of the present invention allows for a smaller diameter of the insertion tube 530.

As hitherto described, the present invention is directed to a capsule type endoscope with an insertion tube comprising a set of mechanisms to ensure maneuverability, adequate operational time, and precise transmission of information.

Accordingly, the capsule type endoscope with an induction tube of the present invention can improve the diagnosis and treatment of the patient by connecting the manipulation wire with the capsule to thereby control the position and the direction of the capsule.

In addition, according to the present invention, it is possible to achieve conspicuous and advantageous effect of using the electric power freely and increasing the use time by connecting the electric power line with the capsule, resulting in the supply of the electric power through a wire.

Moreover, it is possible to transmit images photographed by the capsule to the outside accurately by connecting the signal line with the capsule.

What is claimed is:

1. A capsule type endoscope with an insertion tube, comprising:
a capsule containing a photographic system for acquiring images from the internal cavities of the human body;
at least one manipulation wire connected to the capsule;
an electric power line and a signal line connected to the capsule;
an insertion tube for protecting the manipulation wire(s), the electric power line, and the signal line; and
a connection section for connecting the capsule with the insertion tube.

2. The capsule type endoscope with an insertion tube according to claim 1, wherein the capsule includes:
a signal processing section having at least one CMOS camera;
an illumination section having a light source for irradiating an interior spot or area of the body;
an optical sensor section having an optical system for focal adjustment of images to be photographed by the CMOS camera; and
a signal transmission section having a transmitter for transmitting visual signals obtained by the CMOS camera.

3. The capsule type endoscope with an insertion tube according to claim 1, wherein the manipulation wires are connected to the rear end of the capsule.

4. The capsule type endoscope with an insertion tube according to claim 1, wherein the manipulation wires are made of muscle wire for the connector portion from the capsule to the insertion tube.

5. The capsule type endoscope with an insertion tube according to claim 4, wherein the rest of the manipulation wires are replaced by a single strand of leading wire for conveying electrical signals to the muscle wire.

6. The capsule type endoscope with an insertion tube according to claim 1, wherein the insertion tube is made of flexible material.

7. The capsule type endoscope with an insertion tube according to claim 6, wherein the flexible material is silicon or polyurethane.

8. The capsule type endoscope with an insertion tube according to claim 1, wherein the connection section is made of flexible material so that it can be adequately bent for steering by the manipulation through the manipulation wires.

9. The capsule type endoscope with an insertion tube according to claim 1, wherein a swivel system is constructed at the contact point of the connection section and the capsule.

10. The capsule type endoscope with an insertion tube according to claim 9, having a ball swivel system.

11. The capsule type endoscope with an insertion tube according to claim 1, further comprising a flexible connection tube for protecting the connection section and the manipulation wire.

12. The capsule type endoscope with an insertion tube according to claim 11, wherein the flexible connection tube is made of flexible material in the shape of a corrugated tube.
